SEQUENCE LISTING (32 sequences)

SEQ ID NO: 1 - DNA, 24 bp, Artificial
  Oligonucleotide
ccgacagtaagacgggtaagcctg

SEQ ID NO: 2 - DNA, 22 bp, Artificial
  Oligonucleotide
agcttagtaaagccctcgctag

SEQ ID NO: 3 - DNA, 31 bp, Artificial
  Oligonucleotide
atacgatcgccatggttggatctttggaatc

SEQ ID NO: 4 - DNA, 34 bp, Artificial
  Oligonucleotide
cgatcgtatggatcctcacttgtgagctggacag

SEQ ID NO: 5 - DNA, 100 bp, Artificial
  Oligonucleotide
gcgagtgatttcaccgtcaccggcacaaccgatccgccaaaaagaggcgtaccaatgtcgatatagtcccccgcgcggactgtaggctggagctgcttcg

SEQ ID NO: 6 - DNA, 100 bp, Artificial
  Oligonucleotide
cgccagctattgagcgtcggtatcgatatcggcaccaccaccacccaggtgattttctcccggctggagctggttaaccgcatatgaatatcctccttag

SEQ ID NO: 7 - DNA, 26 bp, Artificial
  Oligonucleotide
gcagaagatcactgtgttggataacg

SEQ ID NO: 8 - DNA, 23 bp, Artificial
  Oligonucleotide
gttcggcatgatgaagcagatgg

SEQ ID NO: 9 - DNA, 99 bp, Artificial
  Oligonucleotide
gccggatgctttctgctccagcatacgtttcgccaaatccacaatgacggctgcggcttcaaccggcggcgtgccgccctgtaggctggagctgcttcg

SEQ ID NO: 10 - DNA, 103 bp, Artificial
  Oligonucleotide
cggcaatgtatatcagtttaaggatgtaaaagaggtgctggctaaagccaacgaactgcgttcgggggatgtgctggcgggcgcatatgaatatcctccttag

SEQ ID NO: 11 - DNA, 24 bp, Artificial
  Oligonucleotide
gcatcaatgccataggtcgcttcc

SEQ ID NO: 12 - DNA, 25 bp, Artificial
  Oligonucleotide
ccggataccttgatttaacgactgg

SEQ ID NO: 13 - DNA, 125 bp, Artificial
  Oligonucleotide
ccactgactttcgccatgacgaaccagaaccagcttagttacagccataatatacctccttattccacacattatacgagccggatgattaattgtcaacagctctgtaggctggagctgcttcg

SEQ ID NO: 14 - DNA, 105 bp, Artificial
  Oligonucleotide
ggttatgcgtaagcattgctgttgcttcgtcgcggcaatataatgagaattattatcattaaaagatgatttgaggagtaagtatcatatgaatatcctccttag

SEQ ID NO: 15 - DNA, 23 bp, Artificial
  Oligonucleotide
ccttcctctttcagcagcttacc

SEQ ID NO: 16 - DNA, 24 bp, Artificial
  Oligonucleotide
cgacgatcagcgcaaagtgaaagg

SEQ ID NO: 17 - DNA, 126 bp, Artificial
  Oligonucleotide
cggcgttccactgcgtttcaccgtggcggactaggtatacctgtaacataatatacctccttattccacacattatacgagccggatgattaattgtcaacagctctgtaggctggagctgcttcg

SEQ ID NO: 18 - DNA, 106 bp, Artificial
  Oligonucleotide
gcgggattggtggtcgcacagacaacttggtgcataatcagcattactcagaaaattaacgttacagcagtatacggaaaaaaagccatatgaatatcctccttag

SEQ ID NO: 19 - DNA, 27 bp, Artificial
  Oligonucleotide
ccttacgaccaatctcatcaataccgg

SEQ ID NO: 20 - DNA, 21 bp, Artificial
  Oligonucleotide
gcaataccatgactcaccagc

SEQ ID NO: 21 - DNA, 100 bp, Artificial
  Oligonucleotide
gtcaggagtattatcgtgattagtctattcgacatgtttaaggtggggattggtccctcatcttcccataccgtagggcctgtaggctggagctgcttcg

SEQ ID NO: 22 - DNA, 103 bp, Artificial
  Oligonucleotide
gggcgagtaagaagtattagtcacactggactttgattgccagaccaccgcgtgaggtttcgcggtatttggcgttcatgtcccatatgaatatcctcctaag

SEQ ID NO: 23 - DNA, 20 bp, Artificial
  Oligonucleotide
cagcgttcgattcatctgcg

SEQ ID NO: 24 - DNA, 20 bp, Artificial
  Oligonucleotide
gaccaatcagcggaagcaag

SEQ ID NO: 25 - DNA, 98 bp, Artificial
  Oligonucleotide
cggcattggcccttccagttctcataccgttggaccaatgaaagcgggtaaacaatttaccgacgatctgattgcccgtgtaggctggagctgcttcg

SEQ ID NO: 26 - DNA, 100 bp, Artificial
  Oligonucleotide
cgcaggcaacgatcttcattgccaggccgccgcgagaggtttcgcggtacttggcgttcatatctttacctgtttcgtaccatatgaatatcctccttag

SEQ ID NO: 27 - DNA, 19 bp, Artificial
  Oligonucleotide
gcgtaagtacagcggtcac

SEQ ID NO: 28 - DNA, 22 bp, Artificial
  Oligonucleotide
cgatgccggaacaggctacggc

SEQ ID NO: 29 - DNA, 35 bp, Artificial
  Oligonucleotide
tgctctagagtccgcgctgtgcaaatccagaatgg

SEQ ID NO: 30 - DNA, 34 bp, Artificial
  Oligonucleotide
cccaagcttaactctctacaacagaaataaaaac

SEQ ID NO: 31 - DNA, 31 bp, Artificial
  Oligonucleotide
ctagtctagattagtacagcagacgggcgcg

SEQ ID NO: 32 - DNA, 35 bp, Artificial
  Oligonucleotide
tcccccgggaagcttccgtcagggcgtggtgaccg

(19) United States
(12) Patent Application Publication
(10) Pub. No.: US 2009/0325245 A1
(43) Pub. Date: Dec. 31, 2009

(54) Ethanolamine Production by Fermentation

(76) Inventors: Philippe Soucaille, Deyme (FR); Rainer Figge, Riom (FR)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/302,726

(22) PCT Filed: Jun. 12, 2007
(86) PCT No.: PCT/EP2007/055762

§ 371 Date: Nov. 26, 2008

(30) Foreign Application Priority Data
Jun. 12, 2006 (EP) PCT/EP2006/063098

(51) Int. Cl. C12P 13/00

(52) U.S. Cl. 435/128

(57) ABSTRACT

The present invention provides a bacterium and a method for the biological production of ethanolamine from a fermentable carbon source. In one aspect of the present invention, a process for the conversion of glucose to ethanolamine is achieved by the use of a recombinant bacterium transformed i) to express a serine decarboxylase enzyme to convert serine to ethanolamine ii) to inactivate the ethanolamine consuming pathways and iii) to increase 3-phosphoglycerate availability. In another aspect of the present invention, the process for the production of ethanolamine from glucose using a recombinant E. coli is improved by i) increasing the flux in the serine pathway and ii) decreasing the flux in the serine consuming pathways.

### FIELD OF INVENTION

The invention comprises a process for the bioconversion of a fermentable carbon source to ethanolamine by an aerobically-grown recombinant bacteria.

### BACKGROUND OF THE INVENTION

Ethanolamine (HOCH₂CH₂NH₂) is the first member of the alpha-hydroxy amine family. Ethanolamine has dual functionality with both alcohol and amine functional groups on a very small molecule that lead in unique chemical attributes.

Ethanolamine is used in i) recovery and removal of acid gases (e.g., carbon dioxide, hydrogen, and hydrogen sulfide) from natural, fuel, and process gas; ii) production of monoalkanolamides for nonionic detergents, emulsifiers, and soaps; iii) synthesis of acelethanolamine, in manufacture of inks, paper, glues, textiles, and polishes; iiii) synthesis of phenylethanolamine for acetate rayon dyes, dyestuffs and iiiii) synthesis of 2-mercaptothiazole in rubber vulcanization acceleration.

Currently more than 600,000 tons of ethanolamine are consumed annually in the United states. It is currently made by a chemical process from ethylene oxide and ammonia.

The biological production of ethanolamine requires the formation of serine as an intermediate which can be decarboxylated to ethanolamine by a plant serine decarboxylase encoded by SDC in Arabidopsis thaliana (Rontein et al, (2001) J. Biol. Chem., 276, 35523-35529). Serine is an amino acid that is used for the production of tryptophan, cysteine, glycine and one-carbon units (Biosynthesis of serine, glycine and one-carbon units, reviewed in Neidhardt, F. C. (Ed. in Chief), R. Curtiss III, J. L. Ingraham, E. C. C. Lin, K. B. Low, B. Magasanik, W. S. Reznikoff, M. Riley, M. Schaechter, and H. E. Umbarger (eds). 1996. Escherichia coli and Salmonella: Cellular and Molecular Biology. American Society for Microbiology).

The glycolytic intermediate 3-phosphoglycerate is converted to serine in three steps. 3-Phosphoglycerate dehydrogenase (serA gene product) oxidizes 3-phosphoglycerate to 3-phosphohydroxypyruvate, the first committed step in the biosynthesis pathway. 3-Phosphoserine aminotransferase (serC gene product) converts 3-phosphohydroxypyruvate to 3-phosphoserine, which is then dephosphorylated to L-serine by 3-phosphoserine phosphatase (serB gene product). Serine is converted to glycine and a C1 unit by serine hydroxymethyltransferase (SHMT) (glyA gene product). Serine can also be converted to pyruvate by serine deaminases encoded by sdaA and sdaB. The flux in the serine pathway is regulated i) at the enzyme level by feed back inhibition of the 3-Phosphoglycerate dehydrogenase and ii) at the genetic level as serA is negatively regulated by the crp-cyclic AMP complex. SerA is also regulated by the leucine-responsive regulatory protein (Lrp) and leucine although Lrp might act indirectly on the serA promoter. On the other hand serB and serC expressions seem to be constitutive.

The problem to be solved by the present invention is the biological production of ethanolamine from an inexpensive carbon substrate such as glucose or other sugars. The number of biochemical steps and the complexity of the metabolic pathways necessitate, for an industrial feasible process of ethanolamine production, the use of a metabolically engineered whole cell catalyst.

### SUMMARY OF THE INVENTION

Applicants have solved the stated problem and the present invention provides bacterium and a method for bioconverting a fermentable carbon source directly to ethanolamine. Glucose is used as a model substrate and recombinant E. coli is used as the model host. In one aspect of this invention, recombinant E. coli expressing a plant serine decarboxylase encoding gene (SDC) converting serine to ethanolamine is constructed. In another aspect of the invention, a recombinant E. coli unable to metabolize ethanolamine is constructed by attenuating the ethanolamine ammonia lyase encoding genes (eutABC). In a further aspect of this invention, the 3-phosphoglycerate availability is increased by attenuating the level of the two phosphoglycerate mutases (encoded by gpmA and gpmB). In a final aspect of the invention the flux in the biosynthesis ethanolamine pathway is increased by increasing the level of 3-Phosphoglycerate dehydrogenase (encoded by serA) and/or phosphoserine aminotransferase (encoded by SerC) and attenuating the level of serine consuming enzymes like serine deaminases (encoded by sdaA and sdaB), serine transacetylase (encoded by cysE), tryptophan synthase (encoded by tprAB) or serine hydroxymethyltransferase (encoded by glyA).

Accordingly it is an object of the present invention to provide a recombinant organism, useful for the production of ethanolamine, comprising one or more of the following characteristics:
(a) a functional serine decarboxylase encoding gene
(b) attenuated genes encoding ethanolamine degrading enzymes, and
(c) an increased availability of the intermediate product 3-phosphoglycerate, obtained by attenuation of the level of expression of phosphoglycerate mutase encoding genes
(d) an increased flux in the serine biosynthesis pathway
(e) attenuated endogenous genes encoding serine consuming enzymes, such as serine deaminases, serine transacetylase, tryptophan synthase or serine hydroxymethyltransferase.

In another embodiment, the invention provides a process for the production of ethanolamine from a recombinant bacterium comprising: (a) contacting the recombinant bacterium of the present invention with at least one renewable carbon source selected from the group consisting of monosaccharides, oligosaccharides, polysaccharides, and single-carbon substrates whereby ethanolamine is produced; and (b) recovering the ethanolamine produced in step (a).

### BRIEF DESCRIPTION OF THE DRAWINGS

The accompanying drawings which are incorporated in and constitute a part of this specification exemplify the invention and together with the description, serve to explain the principles of this invention.

FIG. 1 depicts the genetic engineering of ethanolamine and serine biosynthesis pathways in the development of an ethanolamine producing bacterium from carbohydrates.

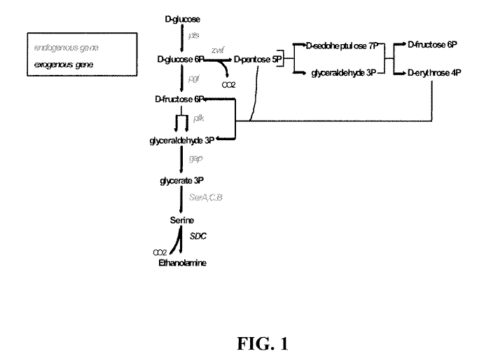

FIG. 2 shows the map of the plasmid pME101-SDCat.

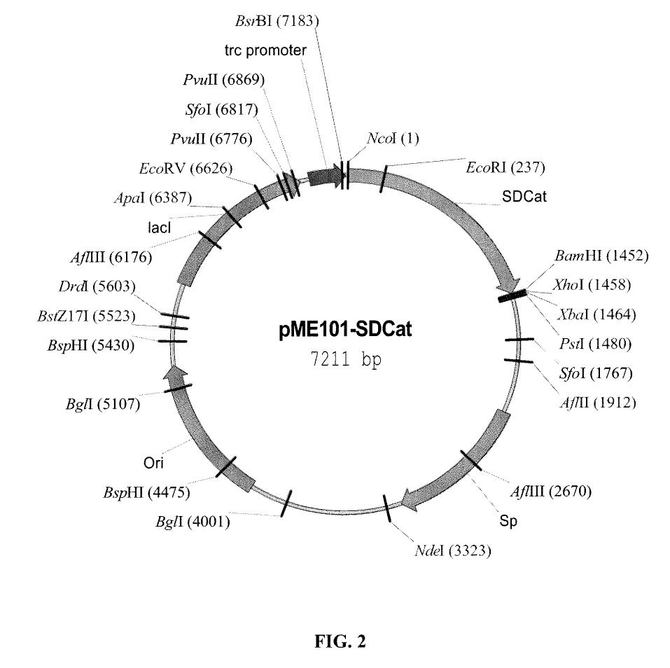

### DETAILED DESCRIPTION OF THE INVENTION

As used herein the following terms may be used for interpretation of the claims and specification.
The term “mutant strain” refers to a non-wild type strain.
The term “bacteria” refers to procaryotic organisms. Bacteria include in particular Enterobacteriaceae, Bacillaceae, Streptomycetaceae and Corynebacteriaceae. Enterobacteriaceae comprise in particular but not exclusively the genera Escherichia, Klebsiella, Salmonella and Pantoea.
The term “transformation” or “transfection” refers to the acquisition of new genes in a cell after the incorporation of nucleic acid. The term “transformant” refers to the product of a transformation. The term “genetically altered” refers to the process of changing hereditary material by transformation or mutation.
The term “expression” refers to the transcription and translation from a gene sequence to the protein, product of the gene.
The term “attenuation” refers to a decrease of expression or activity of a protein, product of the gene of interest. The man skilled in the art knows numerous means to obtain this result, and for example:
Introduction of a mutation into the gene, decreasing the expression level of this gene, or the level of activity of the encoded protein.
Replacement of the natural promoter of the gene by a low strength promoter, resulting in a lower expression.
Use of elements destabilizing the corresponding messenger RNA or the protein
Deletion of the gene if no expression is needed.
   The term “plasmid” or “vector” as used herein refers to an extra chromosomal element often carrying genes which are not part of the central metabolism of the cell, and usually in the form of circular double-stranded DNA molecules.
   The term “carbon substrate” or “carbon source” means any carbon source capable of being metabolized by a bacterium wherein the substrate contains at least one carbon atom. Authors refer particularly to renewable, inexpensive and fermentable carbon sources such as monosaccharides, oligosaccharides, polysaccharides, single-carbon substrates, and polyols such as glycerol. Single carbon substrate are defined as carbon molecules that contain only one carbon atom such as methanol.
   Monosaccharides of the formula (CH₂O)ₙ are also called oses or “simple sugars”; monosaccharides include saccharose, fructose, glucose, galactose and mannose.
   Other carbon sources comprising more than one monosaccharide are called disaccharides, trisaccharides, oligosaccharides and polysaccharides. Disaccharides include saccharose (sucrose), lactose and maltose. Starch and hemicellulose are polysaccharides, also known as “complex sugars”. Therefore, the term “source of carbon” means any product cited above and mixture thereof.
   The term “ATCC” will stand for the American Type Culture Collection, 12301 Parklawn Drive, Rockville, Md. 20852, U.S.A.

In the description of the present invention, enzymes are identified by their specific activities. This definition thus includes all polypeptides that have the defined specific activity also present in other organisms, more particularly in other bacteria. Often enzymes with similar activities can be identified by their grouping to certain families defined as PFAM or COG.

PFAM (protein families database of alignments and hidden Markov models; http://www.sanger.ac.uk/Software/Pfam/) represents a large collection of protein sequence alignments. Each PFAM makes it possible to visualize multiple alignments, see protein domains, evaluate distribution among organisms, gain access to other databases, and visualize known protein structures.

COGs (clusters of orthologous groups of proteins; http://www.ncbi.nlm.nih.gov/COG/) are obtained by comparing protein sequences from 43 fully sequenced genomes representing 30 major phylogenic lines. Each COG is defined from at least three lines, which permits the identification of former conserved domains.

The means of identifying homologous sequences and their percentage homologies are well known to those skilled in the art, and include in particular the BLAST programs, which can be used from the website http://www.ncbi.nlm.nih.gov/BLAST/ with the default parameters indicated on that website. The sequences obtained can then be exploited (e.g., aligned) using, for example, the programs CLUSTALW (http://www.ebi.ac.uk/clustalw/) or MULTALIN (http://prodes.toulouse.inra.fr/multalin/cgi-bin/multalin.pl), with the default parameters indicated on those websites.

Using the references given on GenBank for known genes, those skilled in the art are able to determine the equivalent genes in other organisms, bacterial strains, yeasts, fungi, mammals, plants, etc. This routine work is advantageously done using consensus sequences that can be determined by carrying out sequence alignments with genes derived from other bacteria, and designing degenerate probes to clone the corresponding gene in another organism. These routine methods of molecular biology are well known to those skilled in the art, and are described, for example, in Sambrook et al. (1989 Molecular Cloning: a Laboratory Manual. 2^{nd} ed. Cold Spring Harbor Lab., Cold Spring Harbor, N.Y.).

The present invention provides a method for the fermentative production of ethanolamine, its derivatives or precursors, comprising: culturing a bacterium in an appropriate culture medium comprising a source of carbon and recovering ethanolamine from the culture medium.

In a preferred embodiment, the method is performed with a bacterium which contains at least one gene encoding a polypeptide with serine decarboxylase activity. This gene can be exogenous or endogenous, and can be expressed chromosomally or extrachromosomally. A serine decarboxylase encoding gene can be taken among the SDC genes from plant such as, for example, Arabidopsis thaliana. If needed, a high level of serine decarboxylase activity can be obtained from chromosomally located genes by using one or several copies on the genome that can be introduced by methods of recombination known to the expert in the field. For extrachromosomal genes, different types of plasmids that differ with respect to their origin of replication and thus their copy number in the cell can be used. They may be present as 1-5 copies, 20 copies or up to 500 copies, the figures corresponding to low copy number plasmids with tight replication (pSC101, RK2), low copy number plasmids (pACYC, pRSF110) or high copy number plasmids (pSK bluescript II). The SDC gene may be expressed using promoters with different strength that need or not to be induced by inducer molecules. Examples are the promoters Ptrc, Ptac, Plac, the lambda promoter cI or other promoters known by the expert in the field. Expression of the genes may be boosted by elements stabilizing the corresponding messenger RNA (Carrier and Keasling (1998) Biotechnol. Prog. 15, 58-64) or the protein (e.g. GST tags, Amersham Biosciences).

In another embodiment of this invention, the method is performed with a bacterium wherein the consumption of ethanolamine is decreased, and in particular a bacterium whose expression of genes from the operon eutBC and the gene eutA, encoding the ethanolamine ammonia lyase, has been attenuated. Attenuation of expression of genes can be done by replacing the wild-type promoter by a lower strength promoter, or by the use of an element destabilizing the corresponding messenger RNA or the protein. If needed, complete attenuation of the gene can also be achieved by the deletion of the corresponding DNA sequence coding for the gene. The invention is also specifically related to the bacterium used in this preferred method. The attenuation of the ethanolamine ammonia lyase is especially important, if non-defined media are used for the fermentation, which contain traces of vitamin B12 that can be converted by E. coli to adenosyl-cobalamine the cofactor of the ethanolamine ammonia lyase.

In a further embodiment of the invention, the method is performed with a bacterium whose availability of the intermediate product 3-phosphoglycerate is increased. Preferably, this result is achieved by attenuating the level of expression of genes coding for phosphoglycerate mutases, in particular one or both of gpmA and gpmB genes. This can be done by replacing the wild-type promoter of these genes by a lower strength promoter, or by use of an element destabilizing the corresponding messenger RNA or the protein. The invention is also related to the bacterium used in this particular embodiment of the invention, i.e. a bacterium presenting an increased availability of the 3-phosphoglycerate, in particular a bacterium whose level of expression of the genes coding for phosphoglycerate mutases is attenuated, preferably the level of expression of one or both gpmA and gpmB genes.

In another embodiment, the method is performed with a bacterium whose flux in the serine biosynthesis pathway is stimulated; this result can be achieved by increasing the level of expression of the 3-Phosphoglycerate dehydrogenase and/or phosphoserine aminotransferase, encoded by the serA and serC gene, respectively. Increasing the level of expression of the 3-Phosphoglycerate dehydrogenase and/or phosphoserine aminotransferase can be accomplished by introducing artificial promoters that drive the expression of the serA and/or serC gene, by increasing the number of copies in the cell or by introducing mutations into the serA and/or serC gene that increase the activity of the corresponding protein. The expression of the serA gene can also be increased by replacing the wild type lrp gene (encoding the leucine-responsive regulatory protein) by an lrp mutated allele (such as the lrp-1 allele corresponding to a GLU114ASP substitution in the lrp protein) leading to the constitutive activation of the transcription of the gene serA. The invention is also related to the bacterium used in this particular embodiment of the invention.

In a particular embodiment of the invention mutations can be introduced into the serA gene that reduce its sensitivity to the feed-back inhibitor serine (feed-back desensitized alleles) and thus permit an increased activity in the presence of serine. Examples of desensitized alleles, i.e. feed-back insensitive alleles, have been described in EP 0 931 833 (Ajinomoto) or EP 0 620 853 (Wacker).

In a further embodiment of the invention, the bacterium is modified to present an attenuated level of serine conversion to other compounds than ethanolamine; this result may be achieved by attenuating the level of serine consuming enzymes like serine deaminases (encoded by sdaA and sdaB), serine transacetylase (encoded by cysE), tryptophan synthase (encoded by tprAB) or serine hydroxymethyltransferase (encoded by glyA). Attenuation of these genes can be done by replacing the natural promoter by a lower strength promoter or by element destabilizing the corresponding messenger RNA or the protein. If needed, complete attenuation of the gene can also be achieved by a deletion of the corresponding DNA sequence. The invention is also related to the bacterium used in this particular embodiment of the invention.

In another embodiment, the invention provides a method for the production of ethanolamine with a bacterium, wherein the carbon source is selected from the group consisting of glucose, sucrose, monosaccharides, oligosaccharides, polysaccharides, starch or its derivatives, glycerol and/or single-carbon substrates, and their mixtures thereof.

This invention is also related to a method such as described previously, for the fermentative preparation of ethanolamine, comprising the following steps:
a) Fermentation of an ethanolamine producing bacterium
b) Concentration of ethanolamine in the bacterium or in the medium, and
c) Isolation of ethanolamine from the fermentation broth and/or the biomass, optionally remaining in portions or in the total amount (0-100%) in the end product.

The invention is also related to a bacterium such as defined previously. Preferably, this bacterium is selected among the group consisting of E. coli, C. glutamicum or S. cerevisiae.

Those skilled in the art are able to define the culture conditions for the bacteria according to the invention. In particular the bacteria are fermented at a temperature between 20° C. and 55° C., preferentially between 25° C. and 40° C., and more specifically about 30° C. for C. glutamicum and about 37° C. for E. coli.

The fermentation process is generally conducted in fermenters with an inorganic culture medium of known defined composition adapted to the bacteria used, containing at least one simple carbon source, and if necessary a co-substrate necessary for the production of the metabolite.

### Example 1

### Construction of Strains Expressing a Serine Decarboxylase Encoding Gene: MG1655 (pME101-SDC)

To express a serine decarboxylase enzyme in the host bacterium, the Arabidopsis thaliana SDC gene was expressed from the plasmid pCL1920 (Lerner & Inouye, 1990, NAR 18, 15 p 4631) using the promoter Ptrc. First, for the expression from a low copy vector, the plasmid pME101 was constructed as follows. The plasmid pCL1920 was PCR amplified using the oligonucleotides PME101F and PME101R and the BstZ17I-XmnI fragment from the vector PTRC99A harboring the lacI gene and the P_{trc} promoter was inserted into the amplified vector.

PME101F (SEQ ID NO 1): Ccgacagtaagacgggtaagcctg
PME101R (SEQ ID NO 2): Agcttagtaaagccctcgctag

Then the Arabidopsis thaliana SDC gene was PCR amplified from genomic DNA using the following oligonucleotides:

NcoI SDCatF (SEQ ID NO 3):
Atacgatcgccatggttggatctttggaatc
BamHI SDCatR (SEQ ID NO 4):
CGATCGTATGGATCCTCACTTGTGAGCTGGACAG

The obtained PCR fragment was digested with NcoI and BamHI and cloned into the vector pME101 cut by the same restriction enzymes resulting in plasmid pME101-SDCat.
The pME101-SDCat plasmid was then introduced into the strain MG1655 by usual methods, known by the man skilled in the art.

### Example 2

### Construction of Strains Unable to Metabolize Ethanolamine: MG1655 ΔeutA ΔeutBC (pME101-SDCat)

To delete the eutA gene, the homologous recombination strategy described by Datsenko & Wanner (2000) was used. This strategy allows the insertion of a chloramphenicol or a kanamycin resistance cassette, while deleting most of the genes concerned. For this purpose the following oligonucleotides were used:

DeutAF

(SEQ ID NO 5)

gcgagtgatttcaccgtcaccggcacaaccgatccgccaaaaagaggcgt
accaatgtcgatatagtcccccgcgcggacTGTAGGCTGGAGCTGCTTCG

with
a region (lower case) homologous to the sequence (2563514-2563593) of the gene eutA (reference sequence on the website http://genolist.pasteur.fr/Colibri/),
a region (upper case) for the amplification of the chloramphenicol resistance cassette (reference sequence in Datsenko, K. A. & Wanner, B. L., 2000, PNAS, 97: 6640-6645),

DeutAR

(SEQ ID NO 6)

cgccagctattgagcgtcggtatcgatatcggcaccaccaccacccaggt
gattttctcccggctggagctggttaaccgCATATGAATATCCTCCTTAG

with
a region (lower case) homologous to the sequence (2564895-2564816) of the gene eutA (reference sequence on the website http://genolist.pasteur.fr/Colibri/),
a region (upper case) for the amplification of the chloramphenicol resistance cassette (reference sequence in Datsenko, K. A. & Wanner, B. L., 2000, PNAS, 97: 6640-6645).
   The oligonucleotides DeutAF and DeutAR were used to amplify the chloramphenicol resistance cassette from the plasmid pKD3. The PCR product obtained was then introduced by electroporation into the strain MG1655 (pKD46), in which the Red recombinase enzyme expressed permits the homologous recombination. The chloramphenicol resistant transformants were then selected and the insertion of the resistance cassette is verified by a PCR analysis with the oligonucleotides eutAF and eutAR defined below. The strain retained was designated MG1655 ΔeutA::Cm.

eutAF (SEQ ID NO 7):
gcagaagatcactgtgttggataacg (homologous to the
sequence from 2563130 to 2563155).
eutAR (SEQ ID NO 8):
gttcggcatgatgaagcagatgg (homologous to the
sequence from 2565141 to 2565119).

Then, the eutBC genes deletion was introduced into the strain MG1655 ΔeutA::Cm using the same method as previously described with the following oligonucleotides:

DeutBCF

(SEQ ID NO 9)

gccggatgctttctgctccagcatacgtttcgccaaatccacaatgacgg
ctgcggcttcaaccggcggcgtgccgcccTGTAGGCTGGAGCTGCTTCG

with

a region (lower case) homologous to the sequence (2554448-2554528) of the region of the gene eutC (reference sequence on the website http://genolist.pasteur.fr/Colibri/),

a region (upper case) for the amplification of the kanamycin resistance cassette (reference sequence in Datsenko, K. A. & Wanner, B. L., 2000, PNAS, 97: 6640-6645),

DeutBCR

(SEQ ID NO 10)

Cggcaatgtatatcagtttaaggatgtaaaagaggtgctggctaaagcca
acgaactgcgttcgggggatgtgctggcgggcgCATATGAATATCCTCCT
TAG

with
a region (lower case) homologous to the sequence (2556676-2556594) of the region of the gene eutB (reference sequence on the website http://genolist.pasteur.fr/Colibri/),
a region (upper case) for the amplification of the kanamycin resistance cassette (reference sequence in Datsenko, K. A. & Wanner, B. L., 2000, PNAS, 97: 6640-6645).
   The oligonucleotides DeutBCF and DeutBCR were used to amplify the kanamycin resistance cassette from the plasmid pKD4. The PCR product obtained was then introduced by electroporation into the strain MG1655 ΔeutA::Cm (pKD46). The kanamycin resistant transformants were then selected and the insertion of the resistance cassette was verified by a PCR analysis with the oligonucleotides eutBCF and eutBCR defined below. The strain retained was designated MG1655 ΔeutA::Cm ΔeutBC::Km.

eutBCF (SEQ ID NO 11):
gcatcaatgccataggtcgcttcc (homologous to the
sequence from 2553930 to 2553953).
eutBCR (SEQ ID NO 12):
ccggataccttgatttaacgactgg (homologous to the
sequence from 2556875 to 2556851).

The kanamycin and chloramphenicol resistance cassettes was then be eliminated. The plasmid pCP20 carrying FLP recombinase acting at the FRT sites of the kanamycin and the chloramphenicol resistance cassettes was then introduced into the recombinant sites by electroporation. After a series of cultures at 42° C., the loss of the kanamycin and chloramphenicol resistance cassettes was verified by a PCR analysis with the same oligonucleotides as used previously (eutAF/eutAR and eutBCF/eutBCR). The strain retained was designated MG1655 ΔeutA ΔeutBC.
The pME101-SDCat plasmid was then introduced into the strain MG1655 ΔeutA ΔeutBC.

### Example 3

### Construction of Strains with Decreased Level of 3-Phosphoglycerate: MG1655 ΔeutA ΔeutBC Ptrc18-gpmA Ptrc18-gpmB (pME101-SDCat)

To increase the level of 3-phosphoglycerate, a Ptrc18-gpmA and Ptrc18-gpmB mutants are constructed. First, to reduce the expression of the phosphoglycerate mutase gpmA gene, the promoter is replaced by a modified constitutive trc promoter with weak activity.

The Ptrc-18-gpmA is transferred into the strain MG1655 ΔeutA ΔeutBC by transduction. The MG1655 Ptrc18-gpmA::Km is first constructed using the same method as previously described with the following oligonucleotides

Ptrc18-gpmAF

(SEQ ID NO 13)

CCACTGACTTTCGCCATGACGAACCAGAACCAGCTTAGTTACAGCCATAA
TATACCTCCTTATTCCACACAgTATACGAGCCGGATGATTAATcGcCAAC
AGCTCTGTAGGCTGGAGCTGCTTCG

with
a region (upper case) homologous to the sequence (786771-786819) of the gene gpmA (reference sequence on the website http://genolist.pasteur.fr/Colibri/),
a region (upper bold case) for the amplification of the kanamycin resistance cassette (reference sequence in Datsenko, K. A. & Wanner, B. L., 2000, PNAS, 97: 6640-6645),
a region (upper italic case) for the trc promoter sequence where the −35 and −10 boxes are underlined.

Ptrc18-gpmAR

(SEQ ID NO 14)

ggttatgcgtaagcattgctgttgcttcgtcgcggcaatataatgagaat
tattatcattaaaagatgatttgaggagtaagtatCATATGAATATCCTC
CTTAG

with
a region (lower case) homologous to the sequence (786903-786819) of the region upstream of the gene gpmA (reference sequence on the website http://genolist.pasteur.fr/Colibri/)
a region (upper bold case) for the amplification of the kanamycin resistance cassette (reference sequence in Datsenko, K. A. & Wanner, B. L., 2000, PNAS, 97: 6640-6645).
   The oligonucleotides Ptrc18-gpmAF and Ptrc18-gpmAR are used to amplify the kanamycin resistance cassette from the plasmid pKD4. The obtained PCR product is then introduced by electroporation into the strain MG1655 (pKD46), in which the expressed Red recombinase enzyme permits the homologous recombination. The kanamycin resistant transformants are then selected, and the insertion of the resistance cassette is verified by a PCR analysis with the oligonucleotides gpmAF and gpmAR defined below. The strain retained is designated MG1655 Ptrc18-gpmA::Km.

gpmAF (SEQ ID NO 15):
CCTTCCTCTTTCAGCAGCTTACC (homologous to the
sequence from 786673 to 786695).
gpmAR (SEQ ID NO 16):
cgacgatcagcgcaaagtgaaagg (homologous to the
sequence from 787356 to 787333).

To transfer the modification Ptrc 8-gpmA::Km, a method using a phage P1 transduction is used. The protocol followed is implemented in two steps, with first the preparation of the phage lysate of the strain MG1655 Ptrc18-gpmA::Km, and second the transduction into the strain MG1655 ΔeutA ΔeutBC. The construction of the strain is described above.

1—Preparation of Phage Lysate P1
Inoculation with 100 μl of an overnight culture of the strain MG1655 Ptrc18-gpmA::Km of 10 ml of LB+Km 50 μg/ml+glucose 0.2%+CaCl₂ 5 mM.
Incubation for 30 min at 37° C. with shaking.
Addition of 100 μl of phage lysate P1 prepared on the strain MG1655 (about 1.10⁹ phage/ml).
Shaking at 37° C. for 3 hours until all the cells were lysed.
Addition of 200 μl chloroform and vortexing.
Centrifugation for 10 min at 4500 g to eliminate cell debris.
Transfer of supernatant to a sterile tube and addition of 200 μl chloroform.
Storage of lysate at 4° C.

2—Transduction
Centrifugation for 10 min at 1500 g of 5 ml of an overnight culture of the strain MG1655 ΔeutA ΔeutBC in LB medium.
Suspension of the cell pellet in 2.5 ml of 10 mM MgSO₄, 5 mM CaCl₂
Control tubes: 100 μl cells
   100 μl phages P1 of strain MG1655 Ptrc18-gpmA::Km
Test tube: 100 μl of cells+100 μl of phages P1 of the strain MG1655 Ptrc18-gpmA::Km.
Incubation for 30 min at 30° C. without shaking.
Addition of 100 μl of 1 M sodium citrate in each tube and vortex.
Addition of 1 ml of LB.
Incubation for 1 hour at 37° C. with shaking.
Spreading on dishes LB+Km 50 μg/ml after centrifugation of tubes for 3 min at 7000 rpm.
Incubation at 37° C. overnight.

3—Verification of the Strain

The kanamycin resistant transformants are then selected and the modification of the promoter Ptrc18-gpmA::Km is verified by a PCR analysis with the oligonucleotides gpmAF and gpmAR previously described. The strain retained is designated MG1655 ΔeutA ΔeutBC Ptrc18-gpmA::Km.
Then the Ptrc18-gpmB is transferred into the strain MG1655 ΔeutA ΔeutBC Ptrc18-gpmA::Km by transduction. The MG1655 Ptrc18-gpmB::Cm is first constructed using the same method as previously described with the following oligonucleotides:

Ptrc18-gpmBR

(SEQ ID NO 17)

CGGCGTTCCACTGCGTTTCACCGTGGCGGACTAGGTATACCTGTAACATA
ATATACCTCCTTATTCCACACAgTATACGAGCCGGATGATTAATcGcCAA
CAGCTCTGTAGGCTGGAGCTGCTTCG

with
a region (upper case) homologous to the sequence (4631414-4631366) of the gene gpmB (reference sequence on the website http://genolist.pasteur.fr/Colibri/),
a region (upper bold case) for the amplification of the chloramphenicol resistance cassette (reference sequence in Datsenko, K. A. & Wanner, B. L., 2000, PNAS, 97: 6640-6645),
a region (upper italic case) for the trc promoter sequence where the −35 and −10 boxes are underlined.

Ptrc18-gpmBF

(SEQ ID NO 18)

Gcgggattggtggtcgcacagacaacttggtgcataatcagcattactca
gaaaattaacgttacagcagtatacggaaaaaaagcCATATGAATATCCT
CCTTAG

with
a region (lower case) homologous to the sequence (4631280-4631365) of the region upstream of the gene gpmB (reference sequence on the website http://genolist.pasteur.fr/Colibri/)
a region (upper bold case) for the amplification of the chloramphenicol resistance cassette (reference sequence in Datsenko, K. A. & Wanner, B. L., 2000, PNAS, 97: 6640-6645).
   The oligonucleotides Ptrc18-gpmBF and Ptrc18-gpmBR are used to amplify the chloramphenicol resistance cassette from the plasmid pKD3. The PCR product obtained is then introduced by electroporation into the strain MG1655 (pKD46), in which the Red recombinase enzyme expressed, permits the homologous recombination. The chloramphenicol resistant transformants are then selected and the insertion of the resistance cassette is verified by a PCR analysis with the oligonucleotides gpmBF and gpmBR defined below. The strain retained is designated MG1655 Ptrc18-gpmB::Cm gpmBF (SEQ ID NO 19): ccttacgaccaatctcatcaataccgg (homologous to the sequence from 4630906 to 4630932).

gpmBR (SEQ ID NO 20):
GCAATACCATGACTCACCAGC (homologous to the sequence
from 4631823 to 4631803).

To transfer the modification Ptrc18-gpmB::Cm, the method of phage P1 transduction is used. The preparation of the phage lysate of the strain MG1655 Ptrc18-gpmB::Cm is used for the transduction into the strain MG1655 ΔeutA ΔeutBC Ptrc18-gpmA::Km.

The chloramphenicol resistant transformants are then selected and the Ptrc18-gpmB::Cm is verified by a PCR analysis with the previously defined oligonucleotides gpmBF and gpmBR. The strain retained is designated MG1655 ΔeutA ΔeutBC Ptrc18-gpmA::Km Ptrc18-gpmB::Cm.

The kanamycin and chloramphenicol resistance cassettes can then be eliminated. The plasmid pCP20 carrying FLP recombinase acting at the FRT sites of the kanamycin and the chloramphenicol resistance cassettes is then introduced into the recombinant sites by electroporation. After a series of cultures at 42° C., the loss of the kanamycin and chloramphenicol resistance cassettes is verified by a PCR analysis with the same oligonucleotides as used previously (gpmAF/gpmAR and gpmBF/gpmBR). The strain retained is designated MG1655 ΔeutA ΔeutBC Ptrc18-gpmA Ptrc18-gpmB.
The pME101-SDCat plasmid is then introduced into the strain MG1655 ΔeutA ΔeutBC Ptrc 18-gpmA Ptrc 18-gpmB.

### Example 4

### Construction of Strains with No Serine Deaminase Activity: MG1655 ΔeutA ΔeutBC ΔsdaA ΔsdaB Ptrc18-gpmA Ptrc18-gpmB (pME101-SDCat)

The sdaA gene deletion is introduced into the strain MG1655 ΔeutA ΔeutBC Ptrc18-gpmA Ptrc 8-gpmB by transduction.
The MG1655 ΔsdaA::Km is first constructed using the same method as previously described with the following oligonucleotides:

DsdaAF

(SEQ ID NO 21)

gtcaggagtattatcgtgattagtctattcgacatgtttaaggtggggat
tggtccctcatcttcccataccgtagggccTGTAGGCTGGAGCTGCTTCG

with
a region (lower case) homologous to the sequence (1894941-1895020) of the gene sdaA (reference sequence on the website http://genolist.pasteur.fr/Colibri/),
a region (upper bold case) for the amplification of the kanamycin resistance cassette (reference sequence in Datsenko, K. A. & Wanner, B. L., 2000, PNAS, 97: 6640-6645),

DsdaAR

(SEQ ID NO 22)

GGGCGAGTAAGAAGTATTAGTCACACTGGACTTTGATTGCCAGACCACCG
CGTGAGGTTTCGCGGTATTTGGCGTTCATGTCCCATATGAATATCCTCCT
AAG

with
a region (upper case) homologous to the sequence (1896336-1896254) of the gene sdaA (reference sequence on the website http://genolist.pasteur.fr/Colibri/),
a region (upper bold case) for the amplification of the kanamycin resistance cassette (reference sequence in Datsenko, K. A. & Wanner, B. L., 2000, PNAS, 97: 6640-6645).
   The oligonucleotides DsdaAF and DsdaAR are used to amplify the kanamycin resistance cassette from the plasmid pKD4. The PCR product obtained is then introduced by electroporation into the strain MG1655 (pKD46). The kanamycin resistant transformants are then selected and the insertion of the resistance cassette is verified by a PCR analysis with the oligonucleotides sdaAF and sdaAR defined below. The strain retained is designated MG1655 ΔsdaA::Km.

sdaAF (SEQ ID NO 23):
cagcgttcgattcatctgcg (homologous to the sequence
from 1894341 to 1894360).
sdaAR (SEQ ID NO 24):
GACCAATCAGCGGAAGCAAG (homologous to the sequence
from 1896679 to 1896660).

To transfer the ΔsdaA::Km, the method of phage P1 transduction is used. The preparation of the phage lysate of the strain MG1655 ΔsdaA::Km is used for the transduction into the strain MG1655 ΔeutA ΔeutBC Ptrc18-gpmA Ptrc18-gpmB.
The kanamycin resistant transformants are then selected and the ΔsdaA::Km is verified by a PCR analysis with the previously defined oligonucleotides sdaAF and sdaAR. The strain retained is designated MG1655 ΔeutA ΔeutBC Ptrc18-gpmA Ptrc18-gpmB ΔsdaA::Km. Then the ΔsdaB::Cm is introduced into the strain MG1655 ΔeutA ΔeutBC Ptrc18-gpmA Ptrc18-gpmB ΔsdaA::Km by transduction. The MG1655 ΔsdaB::Cm is first constructed using the same method as previously described with the following oligonucleotides:

DsdaBF

(SEQ ID NO 25)

cggcattggcccttccagttctcataccgttggaccaatgaaagcgggta
aacaatttaccgacgatctgattgcccgTGTAGGCTGGAGCTGCTTCG

with
a region (lower case) homologous to the sequence (2927627-2927705) of the gene sdaB (reference sequence on the website http://genolist.pasteur.fr/Colibri/),
a region (upper bold case) for the amplification of the chloramphenicol resistance cassette (reference sequence in Datsenko, K. A. & Wanner, B. L., 2000, PNAS, 97: 6640-6645),

DsdaBR

(SEQ ID NO 26)

CGCAGGCAACGATCTTCATTGCCAGGCCGCCGCGAGAGGTTTCGCGGTAC
TTGGCGTTCATATCTTTACCTGTTTCGTACCATATGAATATCCTCCTTAG

with
a region (upper case) homologous to the sequence (2928960-2928881) of the gene sdaB (reference sequence on the website http://genolist.pasteur.fr/Colibri/),
a region (upper bold case) for the amplification of the chloramphenicol resistance cassette (reference sequence in Datsenko, K. A. & Wanner, B. L., 2000, PNAS, 97: 6640-6645).
   The oligonucleotides DsdaBF and DsdaBR are used to amplify the chloramphenicol resistance cassette from the plasmid pKD3. The PCR product obtained is then introduced by electroporation into the strain MG1655 (pKD46). The chloramphenicol resistant transformants are then selected and the insertion of the resistance cassette is verified by a PCR analysis with the oligonucleotides sdaBF and sdaBR defined below. The strain retained is designated MG1655 ΔsdaB::Cm.

sdaBF (SEQ ID NO 27):
Gcgtaagtacagcggtcac (homologous to the sequence
from 2927450 to 2927468).
sdaBR (SEQ ID NO 28):
CGATGCCGGAACAGGCTACGGC (homologous to the sequence
from 2929038 to 2929017).

To transfer the ΔsdaB::Cm, the method of phage P1 transduction is used. The preparation of the phage lysate of the strain MG1655 ΔsdaB::Cm is used for the transduction into the strain MG1655 ΔeutA ΔeutBC Ptrc18-gpmA Ptrc18-gpmB ΔsdaA::Km.
The chloramphenicol resistant transformants are then selected and the ΔsdaB::Cm is verified by a PCR analysis with the previously defined oligonucleotides sdaBF and sdaBR. The strain retained is designated MG1655 ΔeutA ΔeutBC Ptrc18-gpmA Ptrc18-gpmB ΔsdaA::Km ΔsdaB::Cm.
The kanamycin and chloramphenicol resistance cassettes can then be eliminated. The plasmid pCP20 carrying FLP recombinase acting at the FRT sites of the kanamycin and the chloramphenicol resistance cassettes is then introduced into the recombinant sites by electroporation. After a series of cultures at 42° C., the loss of the kanamycin and chloramphenicol resistance cassettes is verified by a PCR analysis with the same oligonucleotides as used previously (sdaAF/sdaAR and sdaBF/sdaBR). The strain retained is designated MG1655 ΔeutA ΔeutBC Ptrc18-gpmA Ptrc18-gpmB ΔsdaA ΔsdaB. The pME101-SDCat plasmid is then introduced into the strain MG1655 ΔeutA ΔeutBC Ptrc18-gpmA Ptrc18-gpmB ΔsdaA ΔsdaB.

### Example 5

### Construction of Strains with Increased Serine Pathway Flux

To increase the expression of the serA and serC gene, the gene dosage of the two genes was increased in the ethanolamine producing cell by expressing the enzymes from the copy control vector pCC1BAC (Epicentre) using their own promoters.
For this purpose, first the serC gene was amplified from the E. coli genome using the oligonucleotides Ome 669 and Ome 670. The PCR product was restricted using enzymes XbaI and HindIII and cloned into the vector pUC18 (Stratagene) restricted by the same restriction enzymes. The resulting vector was named pUC18-serC.

Ome 669_serC F (XbaI) (SEQ ID NO 29):
tgcTCTAGAgtccgcgctgtgcaaatccagaatgg

with
a region (lower case) homologous to the sequence (956619-995544) of the gene serC (reference sequence on the website http://genolist.pasteur.fr/Colibri/),
a region (upper case) harbouring the XbaI site

Ome 670_serC R (HindIII) (SEQ ID NO 30):
cccAAGCTTAACTCTCTACAACAGAAATAAAAAC

with
a region (lower case) homologous to the sequence (958028-958004) of the gene serC (reference sequence on the website http://genolist.pasteur.fr/Colibri/)
a region (upper case) harbouring the HindIII site
   Subsequently, the serA gene was amplified from the E. coli genome using the oligonucleotides Ome 621 and Ome 622. The PCR product was restricted using enzymes XbaI and SmaI and cloned into the vector pUC18-serC restricted by the same restriction enzymes. The resulting vector was verified by sequencing and called pUC18-serA-serC.

Ome 621_serA F (XbaI) (SEQ ID NO 31):
CTAGTCTAGAttagtacagcagacgggcgcg

with
a region (lower case) homologous to the sequence (3055198-3055218) of the gene serA (reference sequence on the website http://genolist.pasteur.fr/Colibri/),
a region (upper case) harbouring the XbaI site

Ome 622_serA R (SmaI-HindIII) (SEQ ID NO 32):
TCCCCCGGGaagcttCCGTCAGGGCGTGGTGACCG

with
a region (lower case) homologous to the sequence (3056878-3056859) of the gene serA (reference sequence on the website http://genolist.pasteur.fr/Colibri/),
a region (upper case) harbouring the SmaI and HindIII sites
   To transfer the genes serA and serC into the copy control vector pCC1BAC, the vector pUC18-serA-serC was restricted with the enzyme HindIII and cloned into HindIII cloning ready pCC1BAC (Epicentre).
   The resulting construct was verified and called pCC1BAC-serA-serC. It was transformed into the strain MG1655 (pME101-SDCat).

### Example 6

### Fermentation of Ethanolamine Producing Strains

Strains were initially analyzed in small Erlenmeyer flask cultures using modified M9 medium (Anderson, 1946, Proc. Natl. Acad. Sci. USA 32:120-128, Table 2 below) that was supplemented with 5 g/l MOPS, 5 g/l glucose and 1 mM IPTG. Spectinomycin was added if necessary at a concentration of 50 mg/l. An LB culture was used to inoculate an overnight culture, which in turn was used to inoculate a 50 ml culture to obtain an Optical Density at 600 nm of 0.2. After the culture had reached an OD600 of 1.5 to 2 the culture was stopped and centrifuged.

Glucose and organic acids contents were analyzed by HPLC using a Biorad HPX 97H column for the separation and a refractometer for the detection.

Ethanolamine production was analyzed by GC-MS after derivatization with N-tert-Butyldimethylsilyl-N-methyltrifluoroacetamide (TBDMSTFA).

Serine decarboxylase activity was estimated as follows: cells were resuspended in cold potassium phosphate buffer and sonicated on ice (Branson sonifier, 70W). After centrifugation, proteins contained in the supernatants were quantified (Bradford, 1976). 100 μl of the protein extracts were incubated for 15 minutes at 37° C. with 7.5 mM Serine. The ethanolamine produced by serine decarboxylase activity was quantified by GC-MS after derivatization with TBDMSTFA. Norleucine was included as an internal standard.

Ethanolamine production and serine decarboxylase activity are reported in the table below:

TABLE 1
Ethanolamine production in mmol/gDw and serine
decarboxylase activity (SDC) in mUI/mg protein.

| | Ethanolamine | SDC |
|---|---|---|
| Strain | (mmol/gDw) | (mUI/mg prot) |
| MG1655 | 0.00 | 0.0 |
| MG1655 (pME101-SDCat) | 0.02 | 4.1 |
| MG1655 (pME101-SDCat) | 0.03 | ND |
| (pCC1BAC-serA-serC) | | |
| MG1655 (pME101-SDCat) | 0.02 | ND |
| DeutA DeutBC | | |

ND: not determined.

Strains that produced substantial amounts of metabolites of interest are subsequently tested under production conditions in 300 ml fermentors (DASGIP) using a fed batch protocol.

For this purpose the fermentor is filled with 145 ml of modified minimal medium and inoculated with 5 ml of preculture to an optical density (OD600 nm) between 0.5 and 1.2.

The temperature of the culture is maintained constant at 37° C. and the pH is permanently adjusted to values between 6.5 and 8 using an NH₄OH solution. The agitation rate is maintained between 200 and 300 rpm during the batch phase and is increased to up to 1000 rpm at the end of the fed-batch phase. The concentration of dissolved oxygen is maintained at values between 30 and 40% saturation by using a gas controller. When the optical density reaches a value between 3 and 5, the fed-batch is started with an initial flow rate between 0.3 and 0.5 ml/h and a progressive increase up to flow rate values between 2.5 and 3.5 ml/h. At this point the flow rate is maintained constant for 24 to 48 hours. The medium of the fed is based on minimal media containing glucose at concentrations between 300 and 500 g/l.

TABLE 2
Composition of modified minimal medium M9

| Compound | Concentration | |
|---|---|---|
| ZnSO4•7H2O | 0.0040 | g · L⁻¹ |
| CuCl2•2H2O | 0.0020 | g · L⁻¹ |
| MnSO4•H2O | 0.0200 | g · L⁻¹ |
| CoCl2•6H2O | 0.0080 | g · L⁻¹ |
| H3BO3 | 0.0010 | g · L⁻¹ |
| Na2MoO4•2H2O | 0.0004 | g · L⁻¹ |
| MgSO4•7H2O | 1.00 | g · L⁻¹ |
| CaCl2 2H2O | 0.04 | g · L⁻¹ |
| (NH4)2SO4 | 5.00 | g · L⁻¹ |
| K2HPO4 | 8.00 | g · L⁻¹ |
| Na2HPO4 | 2.00 | g · L⁻¹ |
| (NH4)2HPO4 | 8.00 | g · L⁻¹ |
| NH4Cl | 0.13 | g · L⁻¹ |
| Citric acid | 6.00 | g · L⁻¹ |
| FeSO4, 7H2O | 0.04 | g · L⁻¹ |
| Thiamine | 0.01 | g · L⁻¹ |
| Glucose | 5.00 | g · L⁻¹ |
| Spectinomycine | 0.05 | g · L⁻¹ |

| | |
|---|---|
| NaOH 4M | Adjusted to pH 6.8 |

1. A method for the fermentative production of ethanolamine, its derivatives or precursors, comprising culturing a bacterium in an appropriate culture medium, said medium comprising a source of carbon, and recovering the produced ethanolamine from the culture medium.

2. A method according to claim 1 wherein the bacterium contains at least one gene encoding a polypeptide with serine decarboxylase activity.

3. A method according to claim 2 wherein the polypeptide with serine decarboxylase activity is encoded by a gene from a plant.

4. A method according to claim 3 wherein the plant serine decarboxylase is encoded by SDC from Arabidopsis thaliana.

5. A method according to claim 1, wherein the ethanolamine consuming pathway is attenuated in the bacterium.

6. A method according to claim 5, wherein the ethanolamine ammonia lyase encoding genes (eutBC operon and eutA gene) are attenuated.

7. A method according to claim 1, wherein the bacterium is modified to increase 3-phosphoglycerate availability.

8. A method according to claim 7 wherein 3-phosphoglycerate availability is increased by attenuating the level of expression of one of phosphoglycerate mutases encoding genes.

9. A method according to claim 8 wherein 3-phosphoglycerate availability is increased by attenuating the level of expression of at least one of the genes selected among the group consisting of gpmA and gpmB.

10. A method according to claim 1, wherein the bacterium is transformed to increase the serine pathway flux.

11. A method according to claim 10, wherein the serA encoded protein that is expressed has a reduced sensitivity to serine feed-back inhibition.

12. A method according to claim 10, wherein the level of expression of the serA and/or serC genes is increased.

13. A method according to claim 1, wherein the bacterium is modified to attenuate the serine conversion pathway to compounds other than ethanolamine.

14. A method according to claim 13 wherein the expression of at least one gene selected among the group consisting of:
sdaA encoding serine deaminase
sdaB encoding the second serine deaminase
cysE encoding serine transacetylase
trpAB encoding tryptophane synthase
glyA encoding serine hydroxymethyltransferase is attenuated.

15. A method according to claim 1, wherein the carbon source is chosen among the group consisting of: glucose, sucrose, mono- or oligosaccharides, starch or its derivatives, glycerol, and their mixtures thereof.

16. A method for the fermentative preparation of ethanolamine according to claim 1, comprising the following steps:
a) Fermentation of an ethanolamine producing bacterium
b) Concentration of ethanolamine in the bacterium or in the medium, and
c) Isolation of ethanolamine from the fermentation broth and/or the biomass optionally remaining in portions or in the total amount (0-100%) in the end product.

17. A method for the fermentative production of ethanolamine, its derivatives or precursors, comprising culturing a bacterium in an appropriate culture medium, said medium comprising a source of carbon, and recovering the produced ethanolamine from the culture medium, wherein the bacterium contains at least one gene encoding a polypeptide with serine decarboxylase activity.

18. A method according to claim 17 wherein the polypeptide with serine decarboxylase activity is encoded by a gene from a plant.

19. A method according to claim 18 wherein the plant serine decarboxylase is encoded by SDC from Arabidopsis thaliana.

20. A method according to claim 17 wherein the ethanolamine consuming pathway is attenuated in said bacterium.

21. A method according to claim 17 wherein the bacterium is modified to increase 3-phosphoglycerate availability.

22. A method according to claim 17 wherein the bacterium is transformed to increase the serine pathway flux.

23. A method according to claim 17 wherein the bacterium is modified to attenuate the serine conversion pathway to compounds other than ethanolamine.

24. A method according to claim 17, wherein the carbon source is chosen among the group consisting of: glucose, sucrose, mono- or oligosaccharides, starch or its derivatives, glycerol, and their mixtures thereof.

25. A method for the fermentative preparation of ethanolamine according to claim 17, comprising the following steps:
a) Fermentation of an ethanolamine producing bacterium
b) Concentration of ethanolamine in the bacterium or in the medium, and
c) Isolation of ethanolamine from the fermentation broth and/or the biomass optionally remaining in portions or in the total amount (0-100%) in the end product.

26. A method for the fermentative preparation of ethanolamine, its derivatives or precursors, comprising culturing a bacterium in an appropriate culture medium, said medium comprising a source of carbon, and recovering the produced ethanolamine from the culture medium, wherein said bacterium contains at least one gene encoding a polypeptide with serine decarboxylase activity, and its ethanolamine consuming pathway is attenuated, and said bacterium is modified to increase 3-phosphoglycerate availability, and said bacterium is transformed to increase the serine flux pathway and to attenuate the serine conversion pathway to compounds other than ethanolamine.

27. A method according to claim 26 wherein the polypeptide with serine decarboxylase activity is encoded by a gene from a plant.

28. A method according to claim 27 wherein the plant serine decarboxylase is encoded by SDC from Arabidopsis thaliana.

29. A method according to claim 26 wherein the ethanolamine ammonia lyase encoding genes (eutBC operon and eutA gene) are attenuated.

30. A method according to claim 26 wherein 3-phosphoglycerate availability is increased by attenuating the level of expression of one of phosphoglycerate mutases encoding genes.

31. A method according to claim 30 wherein 3-phosphoglycerate availability is increased by attenuating the level of expression of at least one of the genes selected among the group consisting of gpmA and gpmB.

32. A method according to claim 26 wherein the serA encoded protein that is expressed has a reduced sensitivity to serine feed-back inhibition.

33. A method according to claim 26 wherein the level of expression of the serA and/or serC genes is increased.

34. A method according to claim 26 wherein the expression of at least one gene selected among the group consisting of:
sdaA encoding serine deaminase
sdaB encoding the second serine deaminase
cysE encoding serine transacetylase
trpAB encoding tryptophane synthase
glyA encoding serine hydroxymethyltransferase is attenuated.

35. A method according to claim 26, wherein the carbon source is chosen among the group consisting of: glucose, sucrose, mono- or oligosaccharides, starch or its derivatives, glycerol, and their mixtures thereof.

36. A method for the fermentative preparation of ethanolamine according to claim 26, comprising the following steps:
a) Fermentation of an ethanolamine producing bacterium
b) Concentration of ethanolamine in the bacterium or in the medium, and
c) Isolation of ethanolamine from the fermentation broth and/or the biomass optionally remaining in portions or in the total amount (0-100%) in the end product.
